(19) United States
(12) Patent Application Publication
(10) Pub. No.: US 2007/0148225 A1
(43) Pub. Date: Jun. 28, 2007

(54) PHARMACEUTICAL FORMULATION FOR DELIVERY OF RECEPTOR TYROSINE KINASE INHIBITING (RTKi) COMPOUNDS TO THE EYE

(75) Inventors: Alan L. WEINER, Arlington, TX (US); Malay GHOSH, Fort Worth, TX (US); Wesley Wehsin HAN, Arlington, TX (US)

Correspondence Address: ALCON, IP LEGAL, TB4-8, 6201 SOUTH FREEWAY, FORT WORTH, TX, 76134, US

(73) Assignee: ALCON, INC., Fort Worth, TX (US)

(21) Appl. No.: 11/614,910

(22) Filed: Dec. 21, 2006

Related U.S. Application Data
(60) Provisional application No. 60/753,819, filed on Dec. 23, 2005.

(51) Int. Cl. A61K 9/127; A61K 31/416

(52) U.S. Cl. 424/450; 514/406

(57) ABSTRACT

The present invention relates to development of efficacious pharmaceutical compositions comprising an active compound in a therapeutically effective amount encapsulated or solubilized in phospholipid vesicles.

### BACKGROUND OF THE INVENTION

This application claims priority to U.S. application No. 60/753,819, filed Dec. 23, 2005.

### FIELD OF THE INVENTION

The present invention relates to unique compositions containing compounds with poor solubility and methods useful for treating pathological states that arise or are exacerbated by ocular inflammation, angiogenesis and vascular leakage such as AMD, DR, diabetic macular edema etc., and more specifically, to compositions containing at least one anti-angiogenic agent, anti-inflammatory agent, or anti-vascular permeability agent for use in treating ocular disorders.

### DESCRIPTION OF THE RELATED ARTI

Abnormal neovascularization or angiogenesis and enhanced vascular permeability are major causes for many ocular disorders including age-related macular degeneration is (AMD), retinopathy of prematurity (ROP), ischemic retinal vein occlusions and diabetic retinopathy (DR). AMD and DR are among the most common cause of severe, irreversible vision loss. In these and related diseases, such as retinal vein occlusion, central vision loss is secondary to angiogenesis, the development of new blood vessels from pre-existing vasculature, and alterations in vascular permeability properties.

The angiogenic process is known by the activation of quiescent endothelial cells in pre-existing blood vessels. The normal retinal circulation is resistant to neovascular stimuli, and very little endothelial cell proliferation takes place in the retinal vessels. While there appear to be many stimuli for retinal neovascularization, including tissue hypoxia, inflammatory cell infiltration and penetration barrier breakdown, all increase the local concentration of cytokines (VEGF, PDGF, FGF, TNF, IGF etc.), integrins and proteinases resulting in the formation of new vessels, which then disrupt the organizational structure of the neural retina or break through the inner limiting membranes into the vitreous. Elevated cytokine levels can also disrupt endothelial cell tight junctions, leading to an increase in vascular leakage and retinal edema, and disruption of the organizational structure of the neural retina. Although VEGF is considered to be a major mediator of inflammatory cell infiltration, endothelial cell proliferation and vascular leakage, other growth factors, such as PDGF, FGF, TNF, and IGF etc., are involved in these processes. Therefore, growth factor inhibitors can play a significant role in inhibiting retinal damage and the associated loss of vision upon local delivery in the eye or via oral dosing.

There is no cure for the diseases caused by ocular neovascularization and enhanced vascular permeability. The current treatment procedures of AMD include laser photocoagulation and photodynamic theraphy (PDT). The effects of photocoagulation on is ocular neovascularization and increased vascular permeability are achieved only through the thermal destruction of retinal cells. PDT usually requires a slow infusion of the dye, followed by application of non-thermal laser-light. Treatment usually causes the abnormal vessels to temporarily stop or decrease their leaking. PDT treatment may have to be repeated every three months up to 3 to 4 times during the first year. Potential problems associated with PDT treatment include headaches, blurring, and decreased sharpness and gaps in vision and, in 1-4% of patients, a substantial decrease in vision with partial recovery in many patients. Moreover, immediately following PDT treatment, patients must avoid direct sunlight for 5 days to avoid sunburn. Recently, a recombinant humanized IgG monoclonal antibody fragment was approved (ranibizumab) in the US for treatment of patients with age-related macular degeneration. This drug is typically administered via intravitreal injection once a month.

Many compounds that may be considered potentially useful in treating ocular neovascularization and enhanced vascular permeability-related and other disorders, are poorly soluble in water. A poorly water soluble compound is a substance that is not soluble at a therapeutically effective concentration in an aqueous physiologically acceptable vehicle. Aqueous solubility is an important parameter in formulation development of a poorly water soluble compound. What is needed is a formulation that provides increased solubility of the compound while also providing sufficient bioavailability of the compound so as to maintain its therapeutic potential.

Liposome, lipid based drug carrier vesicle, have emerged as a novel way to deliver, solubilize, and stabilize biologically active compounds. The earliest commercial liposomal formulations in the 1980's were developed for veterinary application (Novasome, IGI, Vineland N.J.) or over the counter cosmetic creams promoted for improved hydration (L'Oreal, Paris and Dior, Paris). More recently, parenteral liposome formulations of amphotericin B, doxorubicin and daunorubicin have been approved and marketed (ABELCET®, The Liposome Co., Inc, Princeton, N.J.; AmBisome®, Gilead Sciences, Foster City, Calif.; DaunoSome™, Nexstar/Fujisawa, Deerfield Park, Ill.; Amphotec®, InterMune, Inc., Brisbane, Calif.; and Doxil®, Sequus/Alza, Menlo Park, Calif.). While the vast majority of liposome preparations are constructed from phospholipids, other non-phospholipid materials can be used either alone or in mixtures to form bilayer arrays. One such example is Amphotec®, which utilizes sodium cholesteryl sulfate as the primary lipid. Other liposome forming materials may include but are not limited to fatty acid compositions, ionized fatty acids, or fatty acyl amino acids, long chain fatty alcohols plus surfactants, ionized lysophospholipids or combinations, non-ionic or ionic surfactants and amphiphiles, alkyl maltosides, αtocopherol esters, cholesterol esters, polyoxyethylene alkyl ethers, sorbitan alkyl esters and polymerized phospholipid compositions.

The present invention provides safe and effective formulations for ocular administration of poorly soluble compounds for the treatment of ocular diseases caused by endothelial cell proliferation, vascular leakage, inflammation and angiogenesis.

### SUMMARY OF THE INVENTION

The present invention overcomes these and other drawbacks of the prior art by providing compositions for treating ocular disorders due to angiogenesis, enhanced endothelial cell proliferation, inflammation, or increased vascular permeability. Within one aspect of the present invention, compositions are provided wherein a compound having poor water solubility is solubilized in phospholipid vesicles for delivery to the eye. The preferred concentration of the phospholipid in the formulation is from 0.05% to 27%.

In another embodiment, posterior juxtascleral (PJ) and periocular liposome formulations containing (a) an active agent, (b) suitable amount of phospholipid such as DMPC, (c) appropriate buffer, and (d) tonicity agent are described. A wide variety of molecules may be utilized within the scope of present invention, especially those molecules having very low solubility. As used herein, the term “poor solubility” is used to refer to a compound having a solubility in water of less than 10 microgram/mL. The active agent for use in the compositions of the invention may be an anti-angiogenic agent, an anti-inflammatory agent, or an anti-vascular permeability agent, or any other poorly water soluble active agent useful for treating ocular disorders.

The amount of phospholipids in the composition plays a very important role in the vesicle shapes and the solubility of the active compound. Optimum concentration of the lipid and drug is 5/1 (micomolar), which provides good multilamellar vesicles (MLV) and large unilamellar vesicles (LUV).

The compositions of the present invention are preferably administered to the eye of a patient suffering from a disorder characterized by neovascularization, inflammation, angiogenesis, or vascular permeability, via posterior juxtascleral administration, intravitreal injection, or topical ocular administration.

### BRIEF DESCRIPTION OF THE DRAWINGS

The following drawings form part of the present specification and are included to further demonstrate certain aspects of the present invention. The invention may be better understood by reference to this drawing in combination with the detailed description of specific embodiments presented herein.

FIG. 1 shows the effects of single intravitreal injection of a receptor tyrosine kinase inhibitor (RTKi) (1%) solubilized in phospholipid vesicles against preretinal neovascularization in the rat Oxygen Induced Retinopathy (OIR) model. The liposome formulation of RTKi completely inhibits preretinal neovascularization.

FIG. 2 shows the effects of single intravitreal injection of a receptor tyrosine kinase inhibitor, RTKi (0.3% and 0.1%) solubilized in phospholipid vesicles against preretinal neovascularization in the rat Oxygen Induced Retinopathy (OIR) model. Both liposome formulations showed statistically significant inhibition of preretinal neovascularization compared to vehicles.

FIG. 3 shows dissected rat retina treated with placebo liposome vehicle. Significant neovascularization is observed in absence of RTKi.

FIG. 4 shows dissected rat retina treated with RTKi (1%) liposome formulation. Complete inhibition of preretinal neovascularization is observed after one intravitreal injection.

### DETAILED DESCRIPTION PREFERRED EMBODIMENTS

As noted above, the present invention provides compositions that contain an active agent having poor water solubility, for use in the treatment of ocular disorders caused by endothelial cell proliferation, enhanced vascular permeability, inflammation, or angiogenesis. The compositions of the invention are useful in treating disorders associated with microvascular pathology, increased vascular permeability and intraocular neovascularization, including diabetic retinopathy (DR), age-related macular degeneration (AMD) and retinal edema.

Briefly, within the context of the present invention, active agents should be understood to be any molecule, either synthetic or naturally occurring, which acts to inhibit vascular growth, reduce vascular permeability, and/or decrease inflammation. In particular, the present invention provides compositions comprising an insoluble or poorly soluble, active agent in a therapeutically effective amount encased in, or solubilized into, phospholipids based vesicles, or liposomes, for ophthalmic use.

A liposome is defined as a structure of one or more concentric spheres of lipid bilayers separated by water or buffer component. These microscopic and spherical vesicles with diameters ranging from 80 nm to 100 μm are formed when hydrated phospholipids arrange themselves in circular sheets with consistent head-tail orientation. These sheets join others to form bilayer membranes that encircles some of the water and water soluble materials in a phospholipid sphere. Liposomes are composed of nontoxic, biodegradable lipids, in particular phospholipids. Efforts were given to prepare liposomes from non-phospholipid compounds that have potential to form lipid bilayer. Methods of liposome preparations and their applications are well known in the art. (See e.g., Weiner 1987; Weiner et al. 1989; Martin 1990; Shek 1995; Ostro 1987; Janoff 1998; Lasic et al. 1998; Barenholtz et al. 1993; and Weiner 1994).

It is contemplated that any active agent that is poorly water soluble may be included in the compositions of the present invention. For example, anti-angiogenic agents, anti-inflammatory agents, or anti-vascular permeability agents are useful in the compositions of the invention.

Preferred anti-angiogenic agents include, but are not limited to, receptor tyrosine kinase inhibitors (RTKi), in particular, those having a multi-targeted receptor profile such as that described in further detail herein; angiostatic cortisenes; MMP inhibitors; integrin is inhibitors; PDGF antagonists; antiproliferatives; HIF-1 inhibitors; fibroblast growth factor inhibitors; epidermal growth factor inhibitors; TIMP inhibitors; insulin-like growth factor inhibitors; TNF inhibitors; antisense oligonucleotides; etc. and prodrugs of any of the aforementioned agents. The preferred anti-angiogenic agent for use in the present invention is a receptor tyrosine kinase inhibitor (RTKi). Most preferred are RTKi's with multi-target binding profiles, such as N-[4-(3-amino-1H-indazol-4-yl) phenyl]-N′-(2-fluoro-5-methylphenyl) urea, having the binding profile substantially similar to that listed in Table 1. Additional multi-targeted receptor tyrosine kinase inhibitors contemplated for use in the compositions of the present invention are described in U.S. application number 2004/0235892, incorporated herein by reference. As used herein, the term “multi-targeted receptor tyrosine kinase inhibitor” refers to a compound having a receptor binding profile exhibiting selectivity for multiple receptors shown to be important in controlling angiogenesis and enhanced vascular permeability related disorders, such as the profile shown in Table 1, and described in co-pending U.S. application number 2006/0189608, incorporated herein by reference. Most preferably, the compounds for use in the formulations of the present invention will have a receptor binding profile similar to that in Table 1.

TABLE 1
Kinase Selectivity Profile of a RTK Inhibitor

| KDR | FLT1 | FLT4 | PDGFR | CSF1R | KIT | FLT3 | TIE2 | FGFR | EGFR | SRC |
|---|---|---|---|---|---|---|---|---|---|---|
| 4 | 3 | 190 | 66 | 3 | 14 | 4 | 170 | >12,500 | >50,000 | >50,000 |

All data reported as IC50 values for kinase inhibition in cell-free enzymatic assays;
ND denotes no data.
Values determined @ 1 mM ATP.

Other agents which will be useful in the compositions and methods of the invention include anti-VEGF antibody (i.e., bevacizumab or ranibizumab); VEGF trap; siRNA molecules, or a mixture thereof, targeting at least two of the tyrosine kinase receptors having IC₅₀ values of less than 200 nM in Table 1; glucocorticoids (i.e., dexamethasone, fluoromethalone, medrysone, betamethasone, triamcinolone, triamcinolone acetonide, prednisone, prednisolone, hydrocortisone, rimexolone, and pharmaceutically acceptable salts thereof, prednicarbate, deflazacort, halomethasone, tixocortol, prednylidene (21-diethylaminoacetate), prednival, paramethasone, methylprednisolone, meprednisone, mazipredone, isoflupredone, halopredone acetate, halcinonide, formocortal, flurandrenolide, fluprednisolone, fluprednidine acetate, fluperolone acetate, fluocortolone, fluocortin butyl, fluocinonide, fluocinolone acetonide, flunisolide, flumethasone, fludrocortisone, fluclorinide, enoxolone, difluprednate, diflucortolone, diflorasone diacetate, desoximetasone (desoxymethasone), desonide, descinolone, cortivazol, corticosterone, cortisone, cloprednol, clocortolone, clobetasone, clobetasol, chloroprednisone, cafestol, budesonide, beclomethasone, amcinonide, allopregnane acetonide, alclometasone, 21-acetoxypregnenolone, tralonide, diflorasone acetate, deacylcortivazol, RU-26988, budesonide, and deacylcortivazol oxetanone); Naphthohydroquinone antibiotics (i.e., Rifamycin); and NSAIDs (i.e., nepafenac, amfenac).

The insoluble, or poorly soluble, active agents for use in the formulations of the present invention will typically solubilize into the lipophilic membrane portion of the liposome in the formulation, as opposed to being “entrapped” within the aqueous inter-lamellar spaces.

Suitable liposomes for use in the formulations of the present invention generally include those in which the lipid component comprises a stable phospholipid. Preferred phospholipids include 1,2-Dimyristoyl-sn-glycero-3-phosphocholine (DMPC), 1,2-Dilauroyl-sn-glycero-3-phosphocholine (DLPC), 1,2-Dipalmotyl-sn-glycero-3-phosphocholine (DPPC), 1,2-Dioleyoyl-sn-glycero-3-phosphocholine(DOPC), 1,2-Distearoyl-sn-glycero-3-phosphocholine(DSPC), 1,2-Dioctanoyl-sn-glycero-3-phosphocholine(DOPC), 1,2-Dimyristoyl-sn-glycero-3-phosphatidyl ethanolamine (DMPE), 1,2-Dilauroyl-sn-glycero-3-phosphatidyl ethanolamine (DLPE), 1,2-Didodecanoyl-sn-glycero-3-phosphatidyl ethanolamine (DDPE), 1,2-Dimyristoyl-sn-glycero-3-phosphoglycerol (DMPG), 1,2-Dilauroyl-sn-glycero-3-phosphoglycerol (DLPG), Egg Phosphatidylcholine (EPC), Soy-Phosphatidylcholine (SPC). More preferred are 1,2-Dimyristoyl-sn-glycero-3-phosphatidyl ethanolamine (DMPE), Egg Phosphatidylcholine (EPC), Soy-Phosphatidylcholine (SPC) and 1,2- Dimyristoyl-sn-glycero-3-phosphoglycerol (DMPG). More preferred are 1,2-Dipalmotyl-sn-glycero-3-phosphocholine (DPPC), 1,2-Dioleyoyl-sn-glycero-3-phosphocholine (DOPC) and 1,2-Dimyristoyl-sn-glycero-3-phosphocholine (DMPC). Most preferred is 1,2- Dimyristoyl-sn-glycero-3-phosphocholine (DMPC).

1,2-Dimyristoyl-sn-glycero-3-phosphocholine (DMPC), is a synthetic phospholipid and is commercially available with purity over 99%. It is a white powder that readily dissolves in ethyl alcohol, chloroform and dichloromethane. Absence of unsaturated functionalities in the compound contributed to its excellent stability.

The liposome formulations of the present invention provide a number of advantages over conventional formulations. One advantage of the present invention is that the formulation containing liposome encapsulated, or solubilized, active agents can successfully solubilize poorly water soluble, or insoluble, compounds, allowing the preparation of an ophthalmologically acceptable and efficacious formulation for local ocular delivery. For example, a liposomal formulation of a receptor tyrosine kinase (RTK) inhibitor, N-[4-(3-amino-1H-indazol-4-yl) phenyl]-N′-(2-fluoro-5-methylphenyl) urea, exhibited 100% inhibition of preretinal neovascularization in rat OIR model.

Another advantage of the encapsulated formulations of the present invention is that they provide a convenient means of slow drug release from an inert depot. In that regard, the liposome formulations of the present invention are completely biodegradable and non-toxic. Furthermore, lipid encapsulation can protect drug from metabolic degradation and the preparation can be injected as a liquid dosage form using a 27-30 gauge needle. The formulations can be sterilized by using standard extrusion methods well known in the art.

The present inventors have discovered that lipid based vesicle formulations can successfully solubilize a highly insoluble active compound. For example, microscopic observations of liposome formulations of the RTKi compound N-[4-(3-amino-1H-indazol-4-yl) phenyl]-N′-(2-fluoro-5-methylphenyl) urea, prepared in DMPC at various concentrations (RTKi/DMPC: 1/5-1/10 micomolar ratio) showed absence of drug crystals, indicating that the drug is soluble in the lipid layer. The present inventors have further observed that the amount of phospholipids used in the formulations of the invention has a profound effect on the vesicle structure and solubilization ability of the micelles. While at higher phospholipids concentration (RTKi/DMPC: 1/7-1/10 micomolar ratio), complete solubilization of RTKi was observed but the vesicles formed were elongated and fused (Table 2). At lower phospholipid concentration (RTKi/DMPC: 1/2-1/4 micomolar ratio) incomplete solubilization was noted as evident from observation of crystals in the formulation. An excellent combination of drug solubilization and formed vesicle structure was achieved using RTKi/DMPC: 1/5 micomolar ratio that provided mostly MLV and LUV vesicles.

TABLE 2

| Drug/DMPC | Microscopic Observation | | |
|---|---|---|---|
| (micomolar | RTKi | | |
| ratio) | Crystal | Vesicle | Vehicle |
| 1/10 | No | Elongated and | Saline* |
| | | fused vesicle | |
| 1/10 | No | Elongated and | Phosphate |
| | | fused vesicle | buffer/pH 7.4 |
| 1/8 | No | Elongated and | Phosphate |
| | | fused vesicle | buffer/pH 7.4 |
| 1/5 | No | Very little | Phosphate |
| | | fused vesicle, | buffer/pH 7.4 |
| | | Mostly MLV | |
| | | and LUV | |
| 1/1 | Yes | MLV and LUV | Phosphate |
| | | | buffer/pH 7.4 |

*pH varies due to lack of buffer

While the preferred phospholipids for use in the compositions of the present invention is DMPC, it is contemplated that other phospholipids may be used either alone or in combination. For example, phospholipids such as 1,2-Dilauroyl-sn-glycero-3-phosphocholine (DLPC), 1,2-Dipalmotyl-sn-glycero-3-phosphocholine (DPPC), 1,2-Dioleyoyl-sn-glycero-3-phosphocholine (DOPC), 1,2-Distearoyl-sn-glycero-3-phosphocholine(DSPC), 1,2-Dioctanoyl-sn-glycero-3-phosphocholine(DOPC), 1,2-Dimyristoyl-sn-glycero-3-phosphatidyl ethanolamine (DMPE), 1,2-Dilauroyl-sn-glycero-3-phosphatidyl ethanolamine (DLPE), 1,2-Didodecanoyl-sn-glycero-3-phosphatidyl ethanolamine (DDPE ), 1,2- Dimyristoyl-sn-glycero-3-phosphoglycerol (DMPG), 1,2-Dilauroyl-sn-glycero-3-phosphoglycerol (DLPG), Egg Phosphatidylcholine (EPC), Soy-Phosphatidylcholine (SPC). More preferred are 1,2-Dimyristoyl-sn-glycero-3-phosphatidyl ethanolamine (DMPE), Egg Phosphatidylcholine (EPC), Soy-Phosphatidylcholine (SoyPC) and 1,2-Dimyristoyl-sn-glycero-3-phosphoglycerol (DMPG). Most preferred is 1,2-Dimyristoyl-sn-glycero-3-phosphoglycerol (DMPG) can be used for this purpose.

In certain preferred embodiments, the formulation of the invention will further comprise a suitable viscosity agent, such as HPMC, HEC, NaCMC, etc. as a dispersant, if necessary. A suitable buffering system, such as phosphate, citrate, borate, tris, etc., may also be used in the formulations of the inventions. Sodium chloride or other tonicity agents may be used to adjust tonicity, if necessary. The composition of the formulation is presented in Table 3. An important feature to obtain “stable” liposome structures that do not rupture is to maintain an osmotic balance across the membrane, i.e. the osmolality on the inside aqueous phases must match the osmolality on the outside. Any method of preparation that produces an osmotic balance across the membrane can be used. This would include methods such as the stable plurilamellar vesicle process, reverse evaporation liposomes, monphasic vesicles, freeze-thaw vesicles, membrane-extruded liposomes, to name a few. Such processes are well-known to the skilled artisan.

TABLE 3

| Ingredients | Amount (w/v, %) |
|---|---|
| RTKi | 0.01-8 |
| Phospholipid (1,2-Dimyristoyl-sn- | 0.05-30 |
| phosphatidylcholine, DMPC) | |
| Dibasic sodium phosphate, dodecahydrate | 0-0.5 |
| Sodium chloride | 0.2-0.9 |
| Viscosity enhancer | 0-0.5% |
| Sodium hydroxide | q.s. to pH |
| Hydrochloric acid | q.s. to pH |
| Water for Injection | q.s. to 100 |

The specific dose level of the active agent for any particular human or animal depends upon a variety of factors, including the activity of the active compound used, the age, body weight, general health, time and route of administration, and the severity of the pathologic condition undergoing therapy.

The formulations described herein may be delivered topically, via intravitreal injection, or via posterior juxtascleral, anterior juxtascleral, and periocular routes. In preferred embodiments of the present invention, for intravitreal and topical applications, the amount of active agent, or poorly water soluble agent will be from about 0.01% to 3%, more preferably from 0.1% to 2% and most preferably from 0.3% to 1%.

Due to the intended route of administration (IVT or PJ), it is very important that the particle size of the formulations must be small to accomplish good syringability, as well as comfort. However, liposome formulations are not like classical solid particle suspensions. Because the particles are in a fluid state they can be significantly larger and still be easily syringable. The largest liposome size currently available is about 40-50 microns, (e.g. SkyePharma's DepoFoam, (Howell 2001)) and it is still very syringable. By definition, liposome suspensions at or below the 100 nm size generally are termed “small unilamellar vesicles (SUV).” Suspensions with particle size from 1m -3 μm are prepared by this compounding procedure. The prepared formulations (for IVT or PJ) exhibit excellent syringibility even when only 2 μL-10 μL of the formulation is injected in the eyes of the animals.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

This example illustrates the preparation of a DMPC based liposome vehicle for intravitreal application.

| Ingredients | Amount (w/v, %) |
|---|---|
| Phospholipid (1,2-Dimyristoyl-sn- | 9 |
| phosphatidylcholine, DMPC) | |
| Dibasic sodium phosphate, dodecahydrate | 0.36 |
| Sodium chloride | 0.8 |
| Sodium hydroxide | q.s. to pH |
| Hydrochloric acid | q.s. to pH |
| Water for Injections | q.s. to 100 |

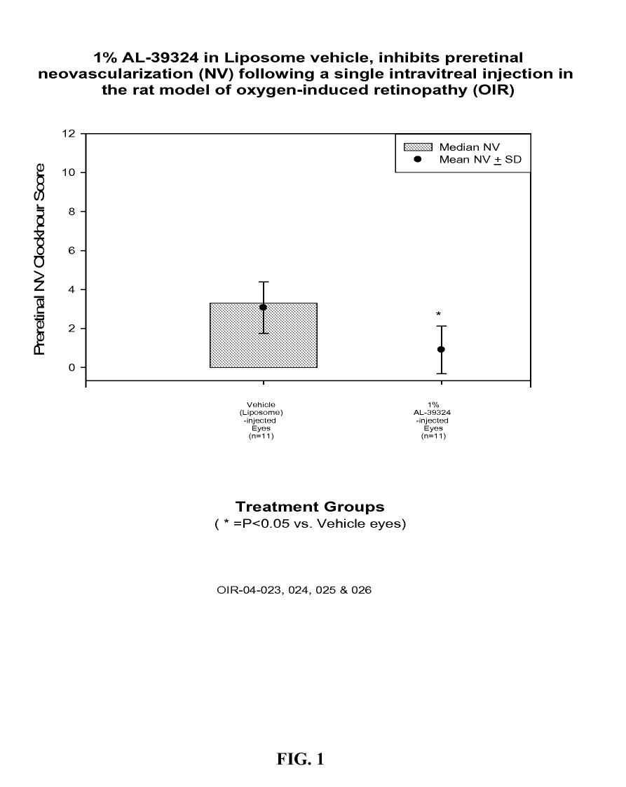

9 g DMPC was dissolved in about 20 mL ethanol. To it was added about 1 g of 0.36% dibasic sodium phosphate solution. Swirl well to make a homogeneous solution. The liquid was removed to a dry thin film by using a rotatory evaporator at 40° C. It was left at vacuum for 4 h. The film was hydrated by addition 80 g of a sterile buffer solution containing 0.36% dibasic sodium phosphate and 0.8% sodium chloride (pH 7.2). Finally q.s to 100 g with the same buffer solution. The solution was stirred at RT for 2 h. This vehicle was injected in rat OIR model and the results are shown in FIG. 1.

### EXAMPLE 2

This example illustrates the preparation of a representative pharmaceutical liposome formulation for intravitreal and topical administration containing a RTKi (N-[4-(3-amino-1H-indazol-4-yl) phenyl]-N′-(2-fluoro-5-methylphenyl) urea).

| Ingredients | Amount (w/v, %) |
|---|---|
| RTKi | 1 |
| Phospholipid (1,2-Dimyristoyl-sn- | 9 |
| phosphatidylcholine, DMPC) | |
| Dibasic sodium phosphate, dodecahydrate | 0.36 |
| Sodium chloride | 0.8 |
| Sodium hydroxide | q.s. to pH |
| Hydrochloric acid | q.s. to pH |
| Water for Injections | q.s. to 100 |

In a 250 mL round bottom flask 1 g sterile RTKi raw material was taken. The compound was dissolved in 20 mL tetrahydrofuran/ethanol (1/5) solvent system. To it was added 9 g DMPC and added another 5 mL ethanol. To the above solution was added 1.5 mL of sterile 0.36% dibasic sodium phosphate, dodecahydrate solution. Swirl well to get a clear colorless solution. The liquid was removed using a rotatory evaporator at 40° C. and left at vacuum for 4 h. Q. s. to 100 g by addition of a buffer solution containing 0.36% dibasic sodium phosphate and 0.8% sodium chloride (pH 7.2). The solution was stirred at RT for 2 h. The above formulation was intravitreally administered in rat OIR model, and the results are shown in FIG. 1.

### EXAMPLE 3

This example illustrates the preparation of a representative pharmaceutical liposome formulation for PJ and periocular administration containing a RTKi (N-[4-(3-amino-1H-indazol-4-yl) phenyl]-N′-(2-fluoro-5-methylphenyl) urea).

| Ingredients | Amount (w/v, %) |
|---|---|
| RTKi | 3 |
| Phospholipid (1,2-Dimyristoyl-sn- | 27 |
| phosphatidylcholine, DMPC) | |
| Dibasic sodium phosphate, dodecahydrate | 0.36 |
| Sodium chloride | 0.7 |
| Sodium hydroxide | q.s. to pH |
| Hydrochloric acid | q.s. to pH |
| Water for Injections | q.s. to 100 |

In a 250 mL round bottom flask 3 g sterile RTKi raw material was taken. The compound was dissolved in 60 mL tetrahydrofuran/ethanol (1/5) solvent system. To it was added 27 g DMPC and added another 5 mL ethanol. To the above solution was added 3.0 mL of sterile 0.36% dibasic sodium phosphate, dodecahydrate solution. Swirl well to get a clear colorless solution. The liquid was then removed at 40° C. using a rotatory evaporator and left at vacuum for 4 h. Q. s. to 100 g by addition of a sterile buffer solution containing 0.36% dibasic sodium phosphate and 0.7% sodium chloride (pH 7.2). The solution was stirred at RT for 2 h.

### EXAMPLE 4

Rat OIR Study: Pregnant Sprague-Dawley rats were received at 14 days gestation and subsequently gave birth on Day 22±1 of gestation. Immediately following parturition, pups were pooled and randomized into separate litters, placed into separate shoebox cages inside oxygen delivery chamber, and subjected to the Double 50 oxygen-exposure profile from Day 0-14 postpartum. Litters were then placed into room air from Day 14/0 through Day 14/6 (days 14-20 postpartum). Additionally on Day 14/0, each pup was randomly assigned to the treatment groups and treatment started from then.

At Day 14/6 (20 days postpartum), all animals were euthanized. Immediately following euthanasia, retinas from all rat pups were harvested, fixed in 10% neutral buffered formalin for 24 hours, subjected to ADPase staining, and fixed onto slides as whole mounts. As the retinas were processed, the success of the vascular staining was confirmed by observation through a dissection scope. A Nikon Eclipse E800® microscope and a Photometrics CoolSNAP fxdigital camera were used to acquire images from each retinal flat mount that was adequately prepared. Computerized image analysis using Metamorph® software was used to obtain a NV clockhour score from each readable sample. Each clockhour out of 12 total per retina was assessed for the presence or absence of preretinal NV. Statistical comparisons using median scores for NV clockhours from each treatment group were utilized in nonparametric analyses. Because the pups were randomly assigned and no difference was observed between the NV scores of control pups from all litters, the NV scores were combined for all treatment groups. P≦0.05 was considered statistically significant.

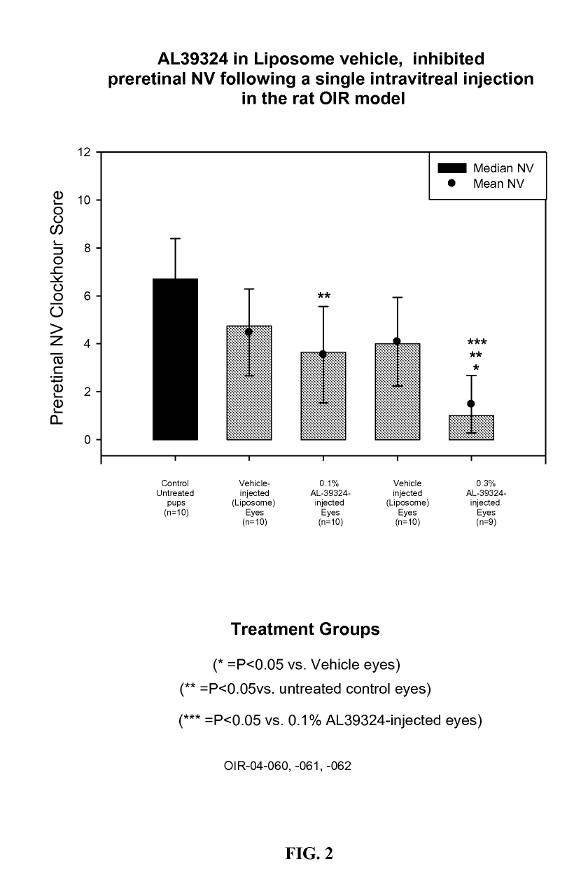

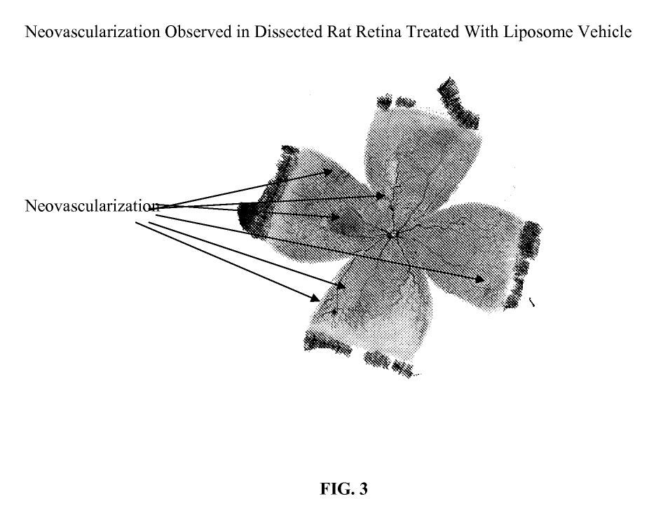

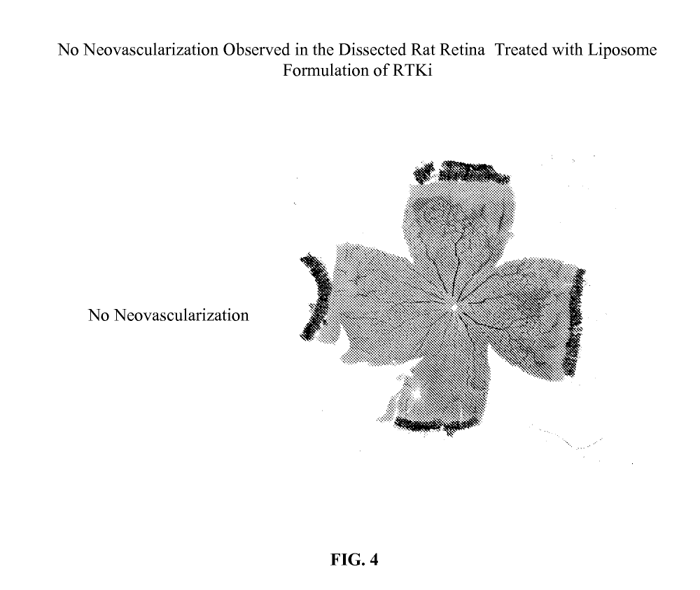

The liposome formulation of RTKi (1% N-[4-(3-amino-1H-indazol-4-yl) phenyl]-N′-(2-fluoro-5-methylphenyl) urea) showed complete (100%) inhibition of preretinal neovascularization in the above described rat OIR model (FIG. 1). About 70% inhibition of preretinal neovascularization was observed in rat OIR model with 0.3% RTKi liposome formulation (FIG. 2). The eyes injected with vehicle or sham injected eyes did not show any inhibition. Dissected rat retina clearly demonstrated that significant neovascularization occurs (FIG. 3) in the rat eyes treated with liposome vehicle containing no drug whereas complete inhibition (FIG. 4) is observed in the rat eyes treated with liposome formulations containing RTKi (1%).

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

United States Patents

Books
Weiner, A. L. Lamellar Systems for Drug Solubilization. In: Liposomes: From Biophysics to Therapeutics, M. J. Ostro, Ed., Marcel Dekker: New York, 1987; pp 339-369
Weiner, A. L.; Cannon, J. B.; Tyle, P. Commercial Approaches to the Delivery of Macromolecular Drugs with Liposomes, In: Controlled Release of Drugs: Polymers and Aggregate Systems, M. Rosoff, Ed., VCH Publishers: New York, 1989; pp. 217-253.
Martin, F. J. Pharmaceutical Manufacturing of Liposomes. In: Specialized Drug Delivery Systems. Manufacturing and Production Technology, P. Tyle, Ed., Marcel Dekker, Inc.: New York, 1990; pp. 267-316.
Shek., P. N., Ed., Liposomes in Biomedical Applications (Drug Targeting and Delivery). Harwood Academic Publishers/Gordon and Breach Publishing Group: Newark, N.J., 1995; Volume 6, 304 p.
Ostro, M. J., Ed. Liposomes: From Biophysics to Therapeutics, Marcel Dekker: New York, 1987, 393 pages
Janoff, A. S., Ed., Liposomes: Rational Design. Marcel Dekker: New York, 1998; 451 p.
Lasic, D. D., Papahadjopoulos, D., Eds., Medical Applications of Liposomes. Elsevier Science Ltd.: Amsterdam, 1998; 779 p.
Barenholtz, Y., Crommelin, D. J. A, Liposomes as Pharmaceutical Dosage Forms, In: Encyclopedia of Pharmaceutical Technology, Swarbrick and Boylan, Eds., Marcel Dekker: New York, 1993; Volume 9, pp 1-39.

Other Publications
S B. Howell, 2001 Clinical Applications of a Novel Sustained-Release Injectable Drug Delivery. System: DepoFoam Technology, Cancer J. 7 (3), 219-225
Weiner, A. L. Liposomes for protein delivery. Immunomethods 1994, 4, 201-209.

We claim:

1. An ophthalmic composition for treating ocular neovascularization, said composition comprising:
at least one poorly water soluble active agent in an amount of from 0.01% to 8%, wherein said active agent is solubilized in phospholipid vesicles.

2. The ophthalmic composition of claim 1, wherein the active agent is selected from the group consisting of anti-angiogenic agents, anti-inflammatory agents, and anti-vascular permeability agents.

3. The ophthalmic composition of claim 2, wherein the active agent is an anti-angiogenic agent.

4. The ophthalmic composition of claim 3, wherein the anti-angiogenic agent is a multi-targeted receptor tyrosine kinase (RTK) inhibitor.

5. The ophthalmic composition of claim 4, wherein the RTK inhibitor is N-[4-(3-amino-iH-indazol-4-yl) phenyl]-N′-(2-fluoro-5-methylphenyl) urea.

6. The ophthalmic composition of claim 1, wherein the concentration of the active agent is from 1% to 3%.

7. The ophthalmic composition of claim 1, wherein the phospholipid is 1,2-Dimyristoyl-sn-glycero-3-phosphocholine (DMPC).

8. The ophthalmic composition of claim 7, wherein the concentration of phospholipid in the formulation is from 2% to 30%

9. A composition for the treatment of ocular neovascularization, said composition comprising from 0. 1 to 3% of a multi-targeted receptor tyrosine kinase (RTK) inhibitor encapsulated in a phospholipid vesicle, wherein said composition is formulated for intravitreal injection into the eye of a patient.

10. The composition of claim 9, wherein the RTK inhibitor is N-[4-(3-amino-1H -indazol-4-yl) phenyl]-N′-(2-fluoro-5-methylphenyl) urea.

11. A composition for the treatment of ocular neovascularization, said composition comprising from 0.5 to 5% of a multi-targeted receptor tyrosine kinase (RTK) inhibitor encapsulated in a phospholipid vesicle, wherein said composition is formulated for posterior juxtascleral administration or periocular administration into the eye of a patient.

12. The composition of claim 11, wherein the RTK inhibitor is N-[4-(3-amino-1H -indazol-4-yl) phenyl]-N′-(2-fluoro-5-methylphenyl) urea.
